(19) United States
(12) Patent Application Publication
(10) Pub. No.: US 2022/0184325 A1
(43) Pub. Date: Jun. 16, 2022

(54) POWDER APPLICATOR

(71) Applicant: SANARA MEDTECH INC., Fort Worth, TX (US)

(72) Inventor: Justin Scruggs, Houston, TX (US)

(21) Appl. No.: 17/550,553

(22) Filed: Dec. 14, 2021

Related U.S. Application Data
(60) Provisional application No. 63/125,476, filed on Dec. 15, 2020.

(51) Int. Cl. A61M 11/00; A61M 31/00; A61M 13/00

(52) U.S. Cl. CPC A61M 11/007; A61M 31/00; A61M 2202/064; A61M 13/00; A61M 11/008

(57) ABSTRACT

An embodiment includes a system comprising a fluid reservoir configured to: (a) inspire fluid when the fluid reservoir expands and expire fluid when the fluid reservoir contracts, and (b) couple to a container that includes a flowable medium. A housing includes first and second channels. A first end of a first conduit couples a second end of the first conduit to the fluid reservoir. The second end of the first conduit is configured to be included in the container when the container is coupled to the fluid reservoir. A first end of a second conduit couples a second end of the second conduit to the housing. The first channel couples the first end of the first conduit to the fluid reservoir. The second channel couples the first end of the second conduit to the container when the container is coupled to the fluid reservoir.

### PRIORITY

This application claims priority to United States Provisional Patent Application No. 63/125,476 filed on Dec. 15, 2021 and entitled “Therapeutic Powder Applicator”, the content of which is hereby incorporated by reference.

### TECHNICAL FIELD

Embodiments of the invention are in the field of powder applicators.

### BACKGROUND

Powders, such as therapeutic powders, are used for various reasons in applications such as, but not limited to, medical applications. For example, such powders may be used as wound dressings, hemostats, and the like. A wound dressing powder, such as hydrolyzed collagen, aids in the natural wound healing process when applied to a wound. The collagen may include bovine collagen and may be indicated for the management of surgical wounds, traumatic wounds, partial- and full-thickness wounds, and/or first- and second-degree burns. The application process for a collagen power may include: (1) prepare the surgical wound site, (2) apply powder to the surgical wound, covering the entire wound bed, and (c) cover the surgical wound as per protocol. A hemostat powder may be used during surgery in order to stop bleeding at the source. Once administered, the blood saturates the powder providing a surface for platelet adhesion and aggregation thus initiating clot formation. This is particularly useful when control of capillary, venous, and arteriolar bleeding by pressure, ligature, and other conventional procedures are ineffective or impractical. Regardless of the form of the powder, to be optimally effective, the powder must be able to be administered in a controlled manner for both volume and placement.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1A includes a cross-sectional view of an embodiment of the invention. FIG. 1B includes a front view of the embodiment of FIG. 1A.

FIG. 2A includes a cross-sectional view of an embodiment of the invention with no container. FIG. 2B includes a front view of the embodiment of FIG. 2A. FIG. 2C includes a perspective view of the embodiment of FIG. 2A.

FIG. 3A includes a front view of an application conduit in an embodiment of the invention. FIG. 3B includes a side view of the embodiment of FIG. 3A. FIG. 3C includes a rear view of the embodiment of FIG. 3A. FIG. 3D includes a cross-sectional view of the embodiment of FIG. 3A. FIG. 3E includes a cross-sectional view of a portion of the embodiment of FIG. 3D.

FIG. 4A includes a side view of a bellows in an embodiment of the invention. FIG. 4B includes a cross-sectional view of a portion of the embodiment of FIG. 4A. FIG. 4C includes a front view of the embodiment of FIG. 4A. FIG. 4D includes a perspective view of the embodiment of FIG. 4A.

FIG. 5A includes a top view of a housing in an embodiment of the invention. FIG. 5B includes a cross-sectional view of the embodiment of FIG. 5A. FIG. 5C includes a side view of the embodiment of FIG. 5A. FIG. 5D includes a rear view of the embodiment of FIG. 5A.

FIG. 6A includes a side view of a snorkel conduit in an embodiment of the invention. FIG. 6B includes a front view of the embodiment of FIG. 6A. FIG. 6C includes a perspective view of the embodiment of FIG. 6A.

FIG. 7A includes a perspective view of an embodiment of the invention. FIG. 7B includes a side view of the embodiment of FIG. 7A. FIG. 7C includes a shadow perspective view of the embodiment of FIG. 7A. FIG. 7D includes a cross-sectional perspective view of the embodiment of FIG. 7A. FIG. 7E includes a cross-sectional view of the embodiment of FIG. 7A. FIG. 7F includes a front view of the embodiment of FIG. 7A.

FIG. 8 includes an embodiment of the invention without a container.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

In order to administer power, such as therapeutic powder, in a controlled manner, Applicant determined conventional systems involve a bellows type device whereby when the bellows are compressed, the expelled air propels the powder out of the device and onto the intended target. A conventional system may include a bellows device that comes pre-loaded with the powder so that once the bellows are depressed, the powder is immediately expelled from the bellows and out of the tip of the device. However, Applicant determined that because the powder is stored inside the bellows, the product delivery is fairly inconsistent. For bellows with pleated walls, this inconsistent delivery may be due to the powder getting trapped inside the folds of the bellows (which then requires the user to shake the device during use in order to dislodge the trapped powder). Other conventional systems are similar except the powder is stored in a separate container and thus avoids the issue of becoming trapped inside the folds of the bellows. Such a system uses a spring-loaded check valve to ensure that the product can only escape when the bellows are depressed. This check valve avoids the powder from entering the bellows.

However, an embodiment allows the powder to be administered from the device without coming into contact with the bellows (thus avoiding the inconsistencies of conventional products while also being able to be administered in nearly any orientation) while avoiding the complexity of spring-loaded check valves used in some conventional products. This is regardless of whether the bellows have pleated or smooth walls. As described herein, through the use of a snorkel tube being placed inside an inverted container that houses the powder, once the bellows are depressed, the compressed air functions to push the powder out of the device via the tip. Upon releasing the bellows, as the bellows expand to draw air back into the device, the powder largely remains stationary at the bottom of the bowl (depending on the orientation in which the device is held) due to gravity. This is true even though, in some embodiments, the entering air must travel through the powder before being drawn back through the snorkel tube. Done in this manner, the powder is largely kept out of the bellows thus allowing for a more consistent application without the need of a spring-loaded mechanism.

There are numerous benefits to embodiments address herein. First, embodiments are less complex than conventional products while maintaining the large majority of functionality of more complex devices. Second, embodiments are superior to conventional products in that they avoid having the powder come into contact with the bellows (thus eliminating issues with consistency of delivery). This is true regardless of whether the bellows has pleated or smoot walls. Third, embodiments allow for the product to be administered directly from the container, thus allowing the option to deliver numerous doses using only one applicator.

An embodiment includes a device that is capable of administering therapeutic powder directly from the container in which it has been packaged. An embodiment may be operable with various sizes of powder containers. For example, various kits may include hydrolyzed collagen powder packaged in 1 g and/or 5 g containers.

An embodiment includes a body that provides receptacles for the tip (sometimes referred to herein as a “second conduit”, bellows, snorkel tube (sometimes referred to herein as a “first conduit”), and a powder container to be attached to the body. With the internal passages of the body configured as shown herein (with a snorkel tube in place), a one-way flow is effectively achieved by preventing the powder (which is denser than air and thus settles to the bottom of the bowl due to gravity) from being sucked back into the bellows as the bellows are recharged. Furthermore, when the bellows are compressed, the pressurized air travels up the snorkel tube and pressurizes the container thus effectively expelling the powder directly from the container and out the tip. An embodiment is a mechanism for delivering the air from the bellows to the powder in the container and overcomes an issue seen in other delivery devices wherein the powder is drawn back into the bellows as it re-expands. An embodiment administers the product directly from the container in which it is supplied, thus allowing for one applicator to be used for multiple containers of product. In contrast, other conventional systems achieve a lesser result in that the powder originates in the bellows of such a device and is thus difficult to get it fully removed resulting in improper dosages and inconsistent applications.

As mentioned above, an embodiment has advantages over conventional technologies in that the powder does not come into contact with the bellows (or does so in a small amount) and thus is prevented from getting trapped in the bellows. This allows for a more accurate dosage and consistent application when compared to conventional systems. Such an embodiment has an advantage over other conventional products in that is simpler in design.

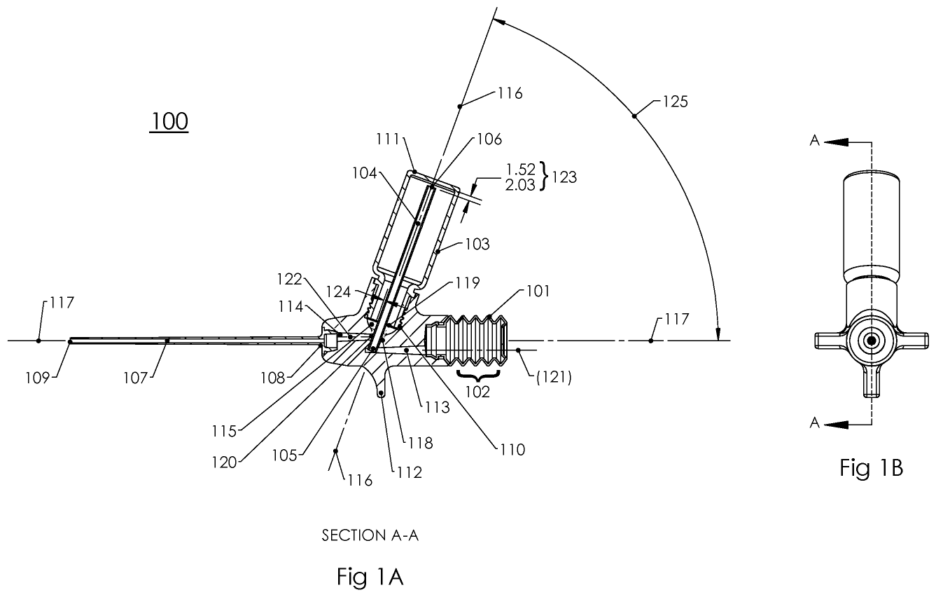

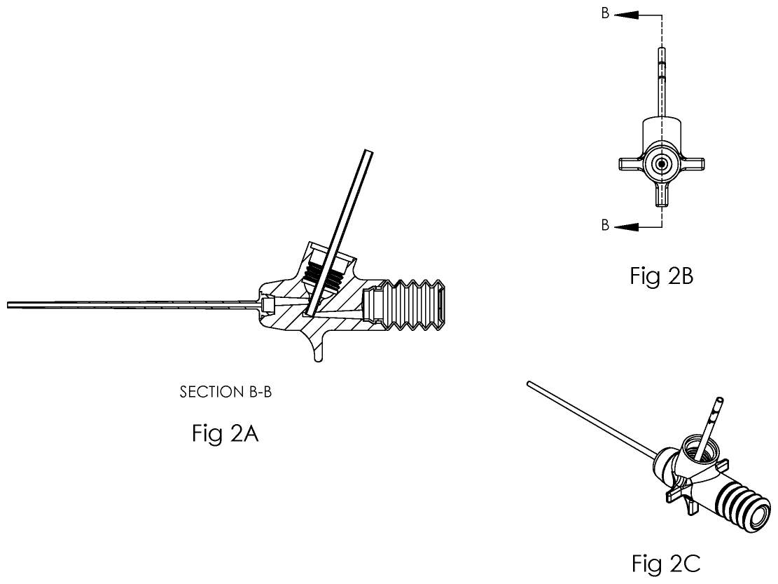

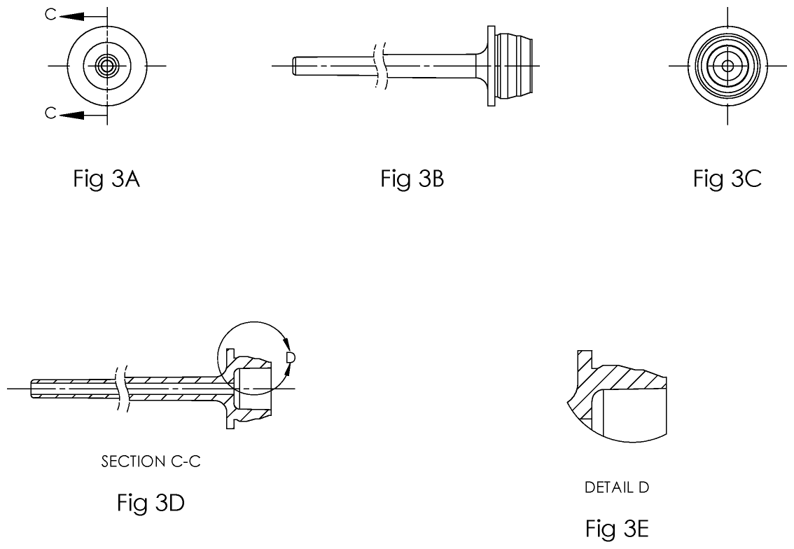

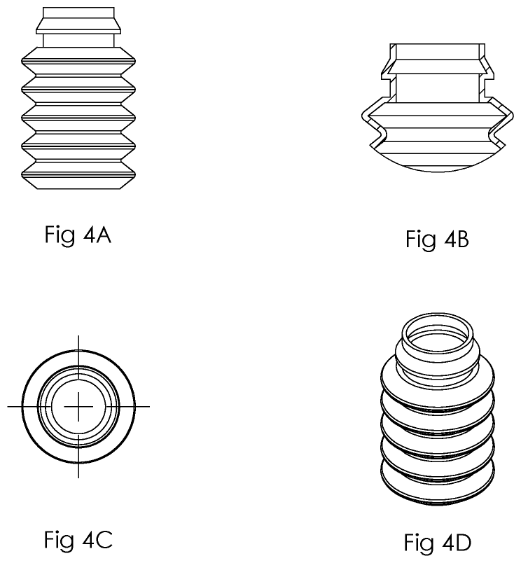

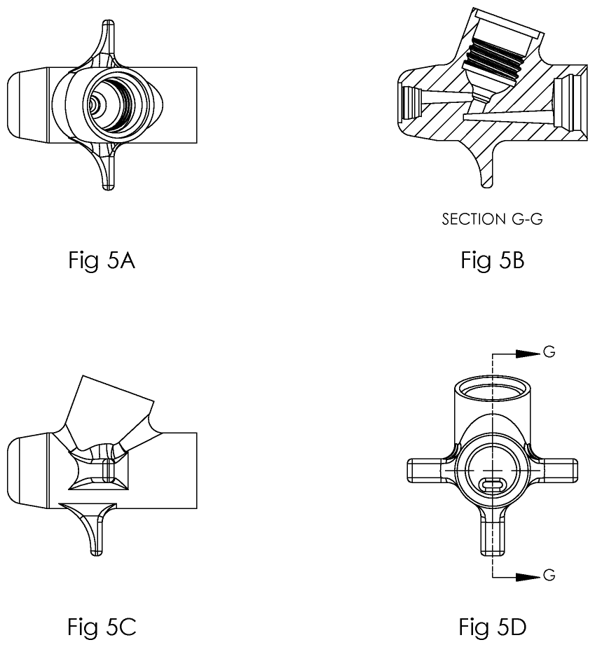

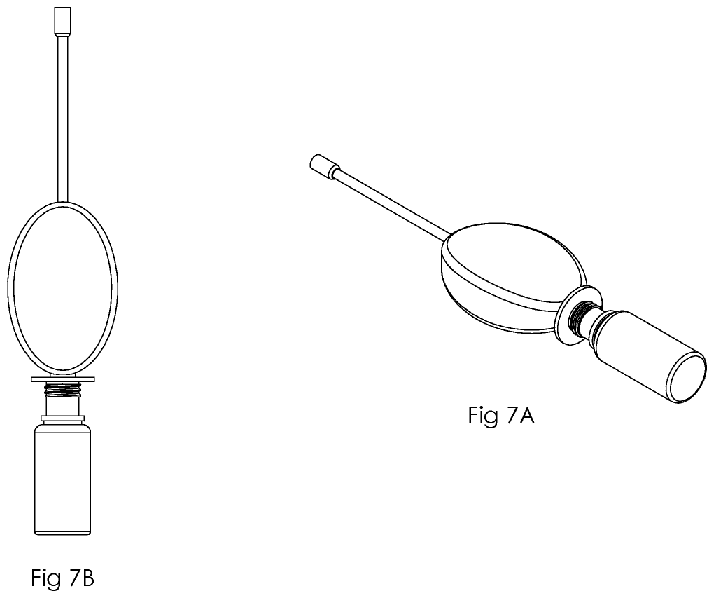

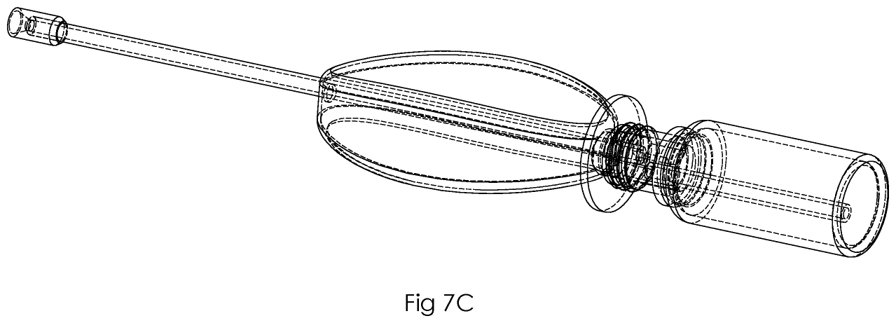

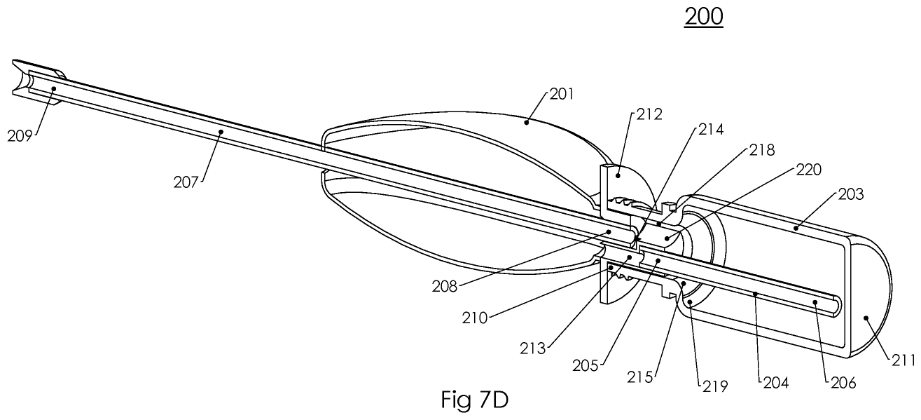

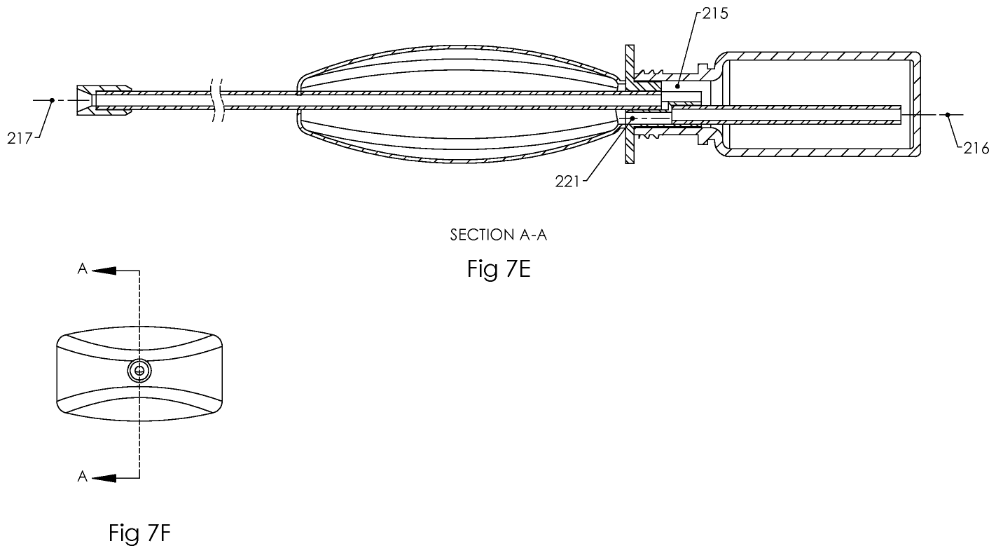

The following examples pertain to further embodiments. Several examples have identifiers. 1XX series identifiers relate to some or all of FIGS. 1A-6C. 2XX series identifiers relate to some or all of FIGS. 7A-7D and/or 8.

For examples 1-20, please see FIGS. 1A-6C.

Example 1. An apparatus (100) comprising: a bellows (101) having a pleated wall (102). The bellows are configured to inspire air when the bellows expand and expire air when the bellows contract. A container (103) includes a powder and is coupled to the bellows. A first conduit (104) is included wherein: (a) a first end (105) of the first conduit couples a second end (106) of the first conduit to the bellows, and (b) the second end of the first conduit is included in the container. A second conduit (107) is included, wherein a first end (108) of the second conduit couples a second end (109) of the second conduit to the container. No powder is included in the bellows. In some embodiments, the powder may be a therapeutic powder.

Alternative version of Example 1. An apparatus (100) comprising: a bellows (101). The bellows are configured to inspire air when the bellows expand and expire air when the bellows contract. A container (103) includes powder and is coupled to the bellows. A first conduit (104) is included wherein: (a) a first end (105) of the first conduit couples a second end (106) of the first conduit to the bellows, and (b) the second end of the first conduit is included in the container. A second conduit (107) is included, wherein a first end (108) of the second conduit couples a second end (109) of the second conduit to the container. No powder is included in the bellows. In some embodiments, the powder may be a therapeutic powder.

Alternative version of Example 1. An apparatus (100) comprising: a fluid reservoir. The fluid reservoir is configured to inspire air when the fluid reservoir expands and expire air when the fluid reservoir contracts. A container (103) includes powder and is coupled to the fluid reservoir. A first conduit (104) is included wherein: (a) a first end (105) of the first conduit couples a second end (106) of the first conduit to the fluid reservoir, and (b) the second end of the first conduit is included in the container. A second conduit (107) is included, wherein a first end (108) of the second conduit couples a second end (109) of the second conduit to the container. No powder is included in the fluid reservoir. In some embodiments, the powder may be a therapeutic powder.

Example 2. The apparatus of example 1, wherein the container includes first (110) and second (111) container ends that oppose each other. The first conduit traverses the first container end but not the second container end.

Example 3. The apparatus of example 2 comprising a housing (112). The housing includes first (113) and second (114) channels. The first channel couples the first end of the first conduit to the bellows. The second channel couples the first end of the second conduit to the container.

Alternative version of Example 3. The apparatus of example 2 comprising a housing (112). The housing includes first (113) and second (114) channels. The first channel couples the first end of the first conduit to the fluid reservoir. The second channel couples the first end of the second conduit to the container.

Example 4. The apparatus of example 3 comprising a funnel (115). The first conduit traverses the funnel.

Example 5. The apparatus of example 4 wherein the apparatus does not include a spring-based valve.

Example 6. The apparatus of example 5 wherein the second end of the first conduit is no more than 5 mm from the second end of the container.

However, in other embodiments the distance may be different. The above is just one example and claims not specifically reciting a specific dimension should not be limited to any specific dimension or range.

Example 7. The apparatus of example 6 wherein the first conduit includes a long central axis (116) that traverses the first and second ends of the first conduit. The second conduit includes a long central axis (117) that traverses the first and second ends of the second conduit. The long central axis of the first conduit in not parallel to the long central axis of the second conduit. The long central axis of the first conduit in not orthogonal to the long central axis of the second conduit.

Example 8. The apparatus of example 7, wherein when the long central axis of the second conduit is in a horizontal plane, the powder is included in the funnel.

In some embodiments, the powder may be a therapeutic powder.

Example 9. The apparatus of example 8, wherein the funnel includes first (118) and second (119) ends that oppose each other. The first end of the funnel has a smaller diameter than the second end of the funnel. The first conduit traverses both of the first and second ends of the funnel. A third channel (120) exists between an outer wall of the first conduit and an inner wall of the first end of the funnel.

Example 10. The apparatus of example 9 comprising a fluid path. The fluid path progresses in serial fashion from the bellows to the first channel, from the first channel to the first conduit, from the first conduit to the container, from the container to the funnel and the third channel, from the third channel to the second channel, and from the second channel to the second conduit.

Another version of Example 10. The apparatus of example 9 comprising a fluid path. The fluid path progresses in serial fashion from the fluid reservoir to the first channel, from the first channel to the first conduit, from the first conduit to the container, from the container to the funnel and the third channel, from the third channel to the second channel, and from the second channel to the second conduit.

Example 11. The apparatus of example 10 wherein: the third channel includes a breadth (124) that extends from the outer wall of the first conduit to the inner wall of the first end of the funnel. The breadth of the third channel is between 0.254 mm and 1.27 mm. In an embodiment, the breadth of the third channel is between 0.6 mm and 0.8 mm. In an embodiment, the breadth of the third channel is between 0.4 mm and 1.0 mm.

However, in other embodiments the distance may be different. The above is just one example and claims not specifically reciting a specific dimension should not be limited to any specific dimension or range.

Example 12. The apparatus of example 11, wherein the third channel extends at least 180 degrees around the first conduit.

Example 13. The apparatus of example 10 wherein: the third channel includes a breadth (124) that extends from the outer wall of the first conduit to the inner wall of the first end of the funnel. The powder includes a plurality of particulates. The breadth of the third channel is based on a breadth of at least one of the particulates.

Example 14. The apparatus of example 13 wherein the breadth of the third channel increases as the breadth of at least one of the particulates increases.

Example 15. The apparatus of example 10 comprising an alternative container that includes additional first and second container ends that oppose each other. The first conduit has a first visual marking and a second visual marking. The first visual marking is configured to be within a distance of the second container end of the container when the apparatus is fully assembled with the container. The second visual marking is configured to be within the distance of the additional second container end of the alternative container when the apparatus is fully assembled with the alternative container. The container has a first volume, the alternative container has a second volume, and the first and second volumes are unequal to one another.

Example 16. The apparatus of example 15 wherein the distance is not greater than 5 mm.

However, in other embodiments the distance may be different. The above is just one example and claims not specifically reciting a specific dimension should not be limited to any specific dimension or range.

Example 17. The apparatus of example 10 comprising: an alternative container that includes additional first and second container ends that oppose each other. An alternative first conduit includes additional first and second ends. The additional second end of the alternative first conduit is no more than 5 mm from the additional second container end of the alternative container when the apparatus is fully assembled with the alternative first conduit and the alternative container. The container has a first volume, the alternative container has a second volume, and the first and second volumes are unequal to one another.

However, in other embodiments the distance may be different. The above is just one example and claims not specifically reciting a specific dimension should not be limited to any specific dimension or range.

Example 18. The apparatus of example 10 wherein the powder includes a therapeutic agent.

The therapeutic agent may include, for example, collagen.

Another version of Example 18. The apparatus of example 10 wherein the powder includes a nacelle structure.

Example 19. The apparatus of example 10 wherein the apparatus does not include a valve that has a movable part.

Example 20. The apparatus of example 10 wherein: the first channel includes a long central axis (121) that traverses first and second ends of the first channel. The second channel includes a long central axis (122) that traverses first and second ends of the second channel. The long central axis of the second conduit in parallel to the long central axes of the first and second channels.

For examples 1a-20a, please see FIGS. 1A-6C.

Example 1a. A system (100) comprising: a bellows (101) having a pleated wall (102). The bellows is configured to inspire a fluid when the bellows expand and expire the fluid when the bellows contract. A container (103) includes powder and is configured to couple to the bellows. The system further includes a first conduit (104). A first end (105) of the first conduit couples a second end (106) of the first conduit to the bellows. The second end of the first conduit is included in the container. The system further includes a second conduit (107). A first end (108) of the second conduit couples a second end (109) of the second conduit to the container.

Another version of Example la. A system (100) comprising a bellows (101). The bellows is configured to inspire a fluid when the bellows expand and expire the fluid when the bellows contract. The system includes a container (103) that includes powder and which is configured to couple to the bellows. The system includes a first conduit (104). A first end (105) of the first conduit couples a second end (106) of the first conduit to the bellows. The second end of the first conduit is included in the container. The system includes a second conduit (107). A first end (108) of the second conduit couples a second end (109) of the second conduit to the container.

Another version of Example la. A system (100) comprising a fluid reservoir, wherein the fluid reservoir is configured to inspire a fluid when the fluid reservoir expands and expire the fluid when the fluid reservoir contracts. The system includes a container (103) that includes powder and which is configured to couple to the fluid reservoir. The system includes a first conduit (104). A first end (105) of the first conduit couples a second end (106) of the first conduit to the fluid reservoir. The second end of the first conduit is included in the container. The system includes a second conduit (107). A first end (108) of the second conduit couples a second end (109) of the second conduit to the container.

Thus, in some embodiments the container is not already coupled to the bellows or fluid reservoir. For example, an embodiment may include a kit where the container is shipped with powder included in the container. The user may unseal the container or generally attach the container to a housing of the system to thereby couple the container to the bellows or fluid reservoir. The kit may include various containers of various sizes. In another embodiment, the housing may be packaged separately from an inventory of containers.

Example 2a. The system of example 1a, wherein: the container includes first (110) and second (111) container ends that oppose each other; and the first conduit traverses the first container end but not the second container end.

Example 3a. The system according to any of examples 1a-2a comprising a housing (112). The housing includes first (113) and second (114) channels. The first channel couples the first end of the first conduit to the bellows. A second channel couples the first end of the second conduit to the container.

Another version Example 3a. The system according to any of examples 1a-2a comprising a housing (112). The housing includes first (113) and second (114) channels. The first channel couples the first end of the first conduit to the fluid reservoir. A second channel couples the first end of the second conduit to the container.

Example 4a. The system according to any of examples 1a-3a comprising a funnel (115), wherein the first conduit traverses the funnel.

Example 5a. The system according to any of examples 1a-4a, wherein the system does not include a spring-based valve.

Example 6a. The system according to any of examples 1a-5a wherein the second end of the first conduit is no more than 3 mm from the second end of the container.

See range 123. In some embodiments, but not all embodiments, this is a critical range. This range needs to be small enough to prevent powder in the container from being sucked into the bellows as the bellow expand. This range helps to form a 1-way valve of sorts without the need to use a complicated spring-based valve or the like. In other embodiments, the range may be less than 1, 2, 4, or 5 mm.

Example 7a. The system according to any of examples 1a-6a wherein: the first conduit includes a long central axis (116) that traverses the first and second ends of the first conduit. The second conduit includes a long central axis (117) that traverses the first and second ends of the second conduit. The long central axis of the first conduit in not parallel to the long central axis of the second conduit. The long central axis of the first conduit in not orthogonal to the long central axis of the second conduit.

See, for example, angle 125. Angle 125 helps contribute to the flow path having a one-way valve of sorts without resorting to a complicated spring-based valve. In an embodiment, angle 125 is less than 90 degrees.

Example 8a. The system according to any of examples 1a-7a, wherein when the long central axis of the second conduit is in a horizontal plane and the container is coupled to the housing, the powder is included in the funnel.

Another version of 8a. The system according to any of examples 1a-7a, wherein the funnel is configured to funnel the powder towards the second channel.

Example 9a. The system according to any of examples 1a-8a, wherein: the funnel includes first (118) and second (119) ends that oppose each other. The first end of the funnel has a smaller diameter than the second end of the funnel. The first conduit traverses both of the first and second ends of the funnel. A third channel (120) exists between an outer wall of the first conduit and an inner wall of the first end of the funnel.

Example 10a. The system according to example 9a comprising a fluid path. The fluid path progresses in serial fashion from the bellows to the first channel, from the first channel to the first conduit, from the first conduit to the container, from the container to the funnel and the third channel, from the third channel to the second channel, and from the second channel to the second conduit.

Example 11a. The system according to any of examples 9a-10a wherein: the third channel includes a breadth (124) that extends from the outer wall of the first conduit to the inner wall of the first end of the funnel. The breadth of the third channel is between 0.254 mm and 1.27 mm. In an embodiment, the breadth of the third channel is between 0.6 mm and 0.8 mm. In an embodiment, the breadth of the third channel is between 0.4 mm and 1.0 mm.

However, in other embodiments the breadth may be different. The above is just one example and claims not specifically reciting a specific dimension or range should not be limited to any specific dimension or range.

See range 124. In some embodiments, but not all embodiments, this is a critical range. This range needs to be small enough such that pressure from the bellows is generally needed to propel powder through channel 114 and into conduit 107. However, the range needs to be large enough such that the powder does not foul or become jammed within this range. In some embodiments the breadth of the third channel is between 0.2 mm and 1.0 mm, 0.1 mm and 1.0 mm, 0.4 mm and 1.2 mm, and the like.

However, in other embodiments the breadth may be different. The above is just one example and claims not specifically reciting a specific dimension or range should not be limited to any specific dimension or range.

Example 12a. The system according to any of examples 9a-11a, wherein the third channel extends at least 180 degrees around the first conduit.

In other embodiments the third channel extends at least 200, 220, 240, 260, 280, 300, 320, 340, or 360 degrees around the first conduit.

Another version of 12a. The system according to any of examples 1a-11a, wherein the third channel is annular and extends 360 degrees around the first conduit.

Example 13a. The system according to any of examples 9a-12a wherein: the third channel includes a breadth (124) that extends from the outer wall of the first conduit to the inner wall of the first end of the funnel. The powder includes a plurality of particulates. The breadth of the third channel is based on a breadth of at least one of the particulates.

Example 14a. The system according to any of examples 9a-13a wherein the breadth of the third channel increases as the breadth of at least one of the particulates increase.

For example, for powders with relatively larger particulates size range 124 may need to be increased to avoid fouling part of the fluid path with the powder. However, for powders with relatively smaller particulates size range 124 may need to be decreased to ensure powder does not flow too freely and without pressure supplied from the bellows.

Example 15a. The system according to any of examples 1a-10a comprising an alternative container that includes additional first and second container ends that oppose each other. The first conduit has a first visual marking and a second visual marking. The first visual marking is configured to be within a distance of the second container end of the container when the system is full assembled with the container. The second visual marking is configured to be within the distance of the additional second container end of the alternative container when the system is full assembled with the alternative container. The container has a first volume, the alternative container has a second volume, and the first and second volumes are unequal to one another.

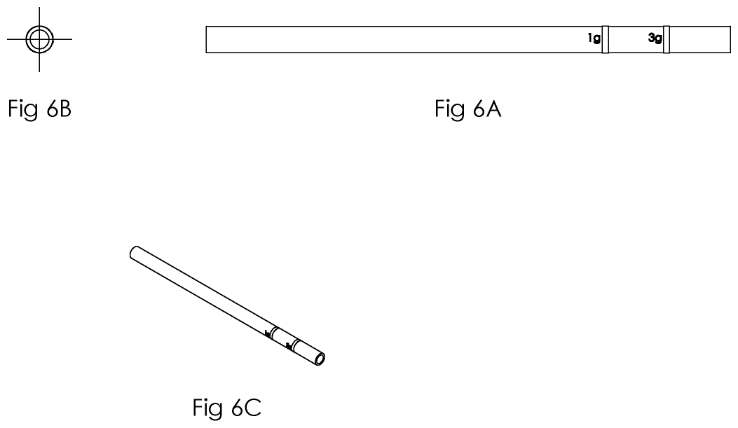

For example, see FIG. 6. The user can simply trim the tube at the line that corresponds to the container size he or she intends to use.

Example 16a. The system according to example 15a wherein the distance is not greater than 5 mm.

However, in other embodiments the distance may be different. The above is just one example and claims not specifically reciting a specific dimension should not be limited to any specific dimension or range.

Example 17a. The system according to any of examples 1a-10a comprising an alternative container that includes additional first and second container ends that oppose each other. An alternative first conduit includes additional first and second ends. The additional second end of the alternative first conduit is no more than 5 mm from the additional second container end of the alternative container when the system is fully assembled with the alternative first conduit and the alternative container. The container has a first volume, the alternative container has a second volume, and the first and second volumes are unequal to one another.

However, in other embodiments the distance may be different. The above is just one example and claims not specifically reciting a specific dimension should not be limited to any specific dimension or range.

In an embodiment instead of having a user trim the first conduit to fit a certain container multiple first conduits can be selected from to match one of several differently sized containers of powder.

Example 18a. The system according to any of examples 1a-10a wherein the powder includes a therapeutic agent.

The therapeutic agent may include, for example, collagen.

Example 19a. The system according to any of examples 1a-10a wherein the system does not include any valve that has a movable part.

Example 20a. The system according to any of examples 1a-10a wherein: the first channel includes a long central axis (121) that traverses first and second ends of the first channel. The second conduit includes a long central axis (122) that traverses first and second ends of the second channel. The long central axis of the second conduit in parallel to the long central axes of the first and second channels.

For examples 1b-4b, please see FIGS. 1A-6C.

Example 1b. A system (100) comprising a bellows (101) having a pleated wall (102). The bellows is configured to inspire a fluid when the bellows expand and expire the fluid when the bellows contract. The system includes a first conduit (104), wherein: (a) a first end (105) of the first conduit couples a second end (106) of the first conduit to the bellows, (b) the second end of the first conduit is configured to be included in a container. The system includes a second conduit (107), wherein a first end (108) of the second conduit couples a second end (109) of the second conduit to a housing (112). The housing includes first (113) and second (114) channels. The first channel couples the first end of the first conduit to the bellows. A second channel is configured to couple the first end of the second conduit to the container.

Another version of Example 1b. A system (100) comprising a bellows (101). The bellows is configured to inspire a fluid when the bellows expand and expire the fluid when the bellows contract. The system includes a first conduit (104), wherein: (a) a first end (105) of the first conduit couples a second end (106) of the first conduit to the bellows, (b) the second end of the first conduit is configured to be included in a container. The system includes a second conduit (107), wherein a first end (108) of the second conduit couples a second end (109) of the second conduit to a housing (112). The housing includes first (113) and second (114) channels. The first channel couples the first end of the first conduit to the bellows. A second channel is configured to couple the first end of the second conduit to the container.

Example 1b. A system (100) comprising a fluid reservoir having a pleated wall (102). The fluid reservoir is configured to inspire a fluid when the fluid reservoir expands and expire the fluid when the fluid reservoir contracts. The system includes a first conduit (104), wherein: (a) a first end (105) of the first conduit couples a second end (106) of the first conduit to the fluid reservoir, (b) the second end of the first conduit is configured to be included in a container. The system includes a second conduit (107), wherein a first end (108) of the second conduit couples a second end (109) of the second conduit to a housing (112). The housing includes first (113) and second (114) channels. The first channel couples the first end of the first conduit to the fluid reservoir. A second channel is configured to couple the first end of the second conduit to the container.

Example 2b. The system of example 1b comprising a plurality of containers, wherein: (a) the plurality of containers includes the container (103), (b) each of the plurality of containers includes powder, and (c) each of the plurality of containers is configured to couple to the bellows.

Example 3b. The system of example 2b wherein the container is fixedly attached to the housing.

Example 4b. The system of example 2b wherein the plurality of containers includes containers having a first volume and containers having a second volume that is unequal to the first volume.

For examples 1c-4c, please see FIGS. 1A-6C.

Example 1c. A system (100) comprising a bellows (101) having a pleated wall (102). The bellows is configured to inspire a fluid when the bellows expand and expire the fluid when the bellows contract. The system includes a first conduit (104), wherein: (a) a first end (105) of the first conduit is to couple a second end (106) of the first conduit to the bellows, (b) the second end of the first conduit is configured to be included in a container. The system includes a second conduit (107), wherein a first end (108) of the second conduit is to couple a second end (109) of the second conduit to a housing (112), wherein: (a) the housing includes first (113) and second (114) channels; (b) the first channel is to couple the first end of the first conduit to the bellows; and (c) a second channel configured to couple the first end of the second conduit to the container.

Another version of Example 1c. A system (100) comprising a bellows (101). The bellows is configured to inspire a fluid when the bellows expand and expire the fluid when the bellows contract. The system includes a first conduit (104), wherein: (a) a first end (105) of the first conduit is to couple a second end (106) of the first conduit to the bellows, (b) the second end of the first conduit is configured to be included in a container. The system includes a second conduit (107), wherein a first end (108) of the second conduit is to couple a second end (109) of the second conduit to a housing (112), wherein: (a) the housing includes first (113) and second (114) channels; (b) the first channel is to couple the first end of the first conduit to the bellows; and (c) a second channel configured to couple the first end of the second conduit to the container.

Example 1c. A system (100) comprising a fluid reservoir. The fluid reservoir is configured to inspire a fluid when the fluid reservoir expands and expire the fluid when the fluid reservoir contracts. The system includes a first conduit (104), wherein: (a) a first end (105) of the first conduit is to couple a second end (106) of the first conduit to the fluid reservoir, (b) the second end of the first conduit is configured to be included in a container. The system includes a second conduit (107), wherein a first end (108) of the second conduit is to couple a second end (109) of the second conduit to a housing (112), wherein: (a) the housing includes first (113) and second (114) channels; (b) the first channel is to couple the first end of the first conduit to the bellows; and (c) a second channel configured to couple the first end of the second conduit to the container.

For instance, some embodiments may include a kit that does not include the container (which may be delivered separately from the housing).

Example 2c. The system of example 1c comprising a plurality of containers, wherein: (a) the plurality of containers includes the container (103), (b) each of the plurality of containers includes powder, and (c) each of the plurality of containers is configured to couple to the bellows.

Another version of Example 2c. The system of example 1c comprising a plurality of containers, wherein: (a) the plurality of containers includes the container (103), (b) each of the plurality of containers includes powder, and (c) each of the plurality of containers is configured to couple to the fluid reservoir.

Example 3c. The system of example 2c wherein the container is fixedly attached to the housing.

Example 4c. The system of example 2c wherein the plurality of containers includes containers having a first volume and containers having a second volume that is unequal to the first volume.

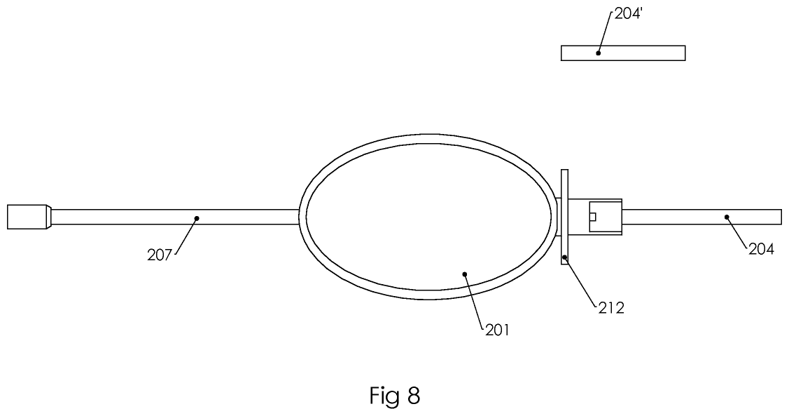

For examples 1d-21d, please see FIGS. 7A-8.

Example 1d. A system (200) comprising a fluid reservoir (201). The fluid reservoir is configured to inspire fluid when the fluid reservoir expands and expire fluid when the fluid reservoir contracts. Accordingly, the fluid reservoir is a type of bellows regardless of whether it has pleated walls. The fluid reservoir is configured to couple to a container (203). For instance, the container may have threads that mate with threads on the fluid reservoir a component to which the fluid reservoir is coupled. The container includes a flowable medium or may be filled fully or partially with a such a medium. The flowable medium may be a gas, powder, liquid, solution, emulsion, or combinations thereof. The system may include a housing (212) including first (213) and second (214) channels. The system may further include a first conduit (204), wherein: (a) a first end (205) of the first conduit couples a second end (206) of the first conduit to the fluid reservoir, and (b) the second end of the first conduit is configured to be included in the container when the container is coupled to the fluid reservoir. The system may include a second conduit (207), wherein a first end (208) of the second conduit couples a second end (209) of the second conduit to the housing. The first channel couples the first end of the first conduit to the fluid reservoir. The second channel couples the first end of the second conduit to the container when the container is coupled to the fluid reservoir. In some embodiments, none of the flowable medium is included in the fluid reservoir. In some embodiments, the system is configured so none of the flowable medium is included in the fluid reservoir. For instance, the end of the first tube is close enough to the end of the container such that, at least in most orientations of the system, the fluid reservoir may expand and compress without sucking medium from the container into the fluid reservoir.

As used herein, a “container” is an object that can be used to hold or transport something (e.g., a bottle, bag).

As used herein, a “flowable medium” is a movable substance. Such a medium may move in any flow pattern, such as turbulent or laminar flow. Such a substance may be in various phases such as a solid, liquid, gas, or combinations thereof.

As used herein, a “conduit” is a pipe, tube, duct, and the like.

As used herein, a “channel” is a gutter, trough, passage, route, tunnel, groove, pass and the like. A conduit is a form of channel.

As used herein, a “fluid” may include a liquid, gas, a suspension of particles in a liquid or gas, aerosols, and the like. Embodiments are not necessarily limited to powders and fluids may be dispersed from such embodiments.

As used herein, a “fluid reservoir” is a basin, container, or bulb to retain fluid.

As used herein, a “bellows” is an instrument or machine that by alternate expansion and contraction draws in air through a valve or orifice and expels it through the valve, orifice or another orifice. A “bellows” does not necessarily have pleated walls.

Example 2d. The system of example 1d comprising a fluid path, wherein the fluid path progresses in serial fashion from the fluid reservoir to the first channel, from the first channel to the first conduit, from the first conduit to a second end of the container when the container is coupled to the fluid reservoir, from the second end of the container when the container is coupled to the fluid reservoir to the second channel, and from the second channel to the second conduit.

This is not to say that a molecule of air in the reservoir necessarily passes from the reservoir all the way through the system and out the tip of the second conduit in order to use the device. Instead, the fluid path may couple different areas of fluid (e.g., air) and medium (e.g., powder) together such that expelling fluid from the reservoir will cause the medium to eject from the container and the second conduit.

Example 3d. The system of example 2d, wherein the system is configured to convey the flowable medium from the container and out the second end of the second conduit when the container is coupled to the fluid reservoir and in response to contraction of the fluid reservoir.

Such “contraction” may include, for example, a user squeezing the reservoir.

Example 4d. The system of example 3d, wherein the housing includes a funnel, and the first conduit traverses the funnel.

Example 5d. The system of example 3d, wherein the container includes a funnel (215) and the first conduit is configured to traverse the funnel when the container is coupled to the fluid reservoir.

Another version of Example 5d. The system of example 3d comprising a funnel and the first conduit is configured to traverse the funnel when the container is coupled to the fluid reservoir.

Thus, in some embodiments the funnel may be included in the system in various ways. For example, the funnel may be monolithic with the container, housing, or neither of the container or housing.

As used herein, “monolithic” means formed of a single portion of material without joints or couplings (e.g., welds, adhesions).

Example 6d. The system of example 5d, wherein the system does not include a valve.

As used herein, a “valve” is a device that regulates, directs or controls the flow of a fluid by opening or closing a passageway by moving a portion of the device between two or more positions.

Another version of Example 6d. The system of example 5d, wherein the system does not include a spring-based valve and the system does not include a valve that has a movable part.

As a result, the system has adequate performance (in terms of applying a medium, such as a powder) while maintaining efficiency in manufacturing.

Example 7d. The system of example 6d, wherein the second end of the first conduit is no more than 5 mm from the second end of the container when the container is coupled to the fluid reservoir.

In other embodiments the second end of the first conduit is no more than 1, 3, 7, 9, 11, 13, 15, 17, 19 mm or more from the second end of the container when the container is coupled to the fluid reservoir. The range can depend on the size of the container holding the flowable medium, the fluid reservoir, and the like.

Such close proximity between the conduit and container end helps prevent backflow of medium (e.g., powder) into the fluid reservoir (where the material may be “wasted” if the use then has difficulty ejecting the medium from the reservoir).

Example 8d. The system of example 6d, wherein the first conduit includes a long central axis (216) that traverses the first and second ends of the first conduit. The second conduit includes a long central axis (217) that traverses the first and second ends of the second conduit. The long central axis of the first conduit is parallel to the long central axis of the second conduit.

Example 9d. The system of example 6d, wherein the funnel includes first (218) and second (219) ends that oppose each other. The first end of the funnel has a smaller diameter than the second end of the funnel. The first conduit traverses both of the first and second ends of the funnel when the container is coupled to the fluid reservoir.

Example 10d. The system of example 6d comprising an alternative first conduit (204′). A first end of the alternative first conduit is configured to couple a second end of the alternative first conduit to the fluid reservoir. The second end of the alternative first conduit is configured to be included in an alternative container when the alternative container is coupled to the fluid reservoir. The first conduit and the alternative first conduit have different lengths from each other. The container and the alternative container have different volumes from each other.

For instance, in FIG. 8 a user may exchange conduit 204 for conduit 204′. Conduit 204′ may be longer or shorter than conduit 204. Conduit 204′ may be configured to be 5 mm or less from the edge of the alternative container when the alternative container is coupled to the system. However, such a long conduit may be too long for a smaller container. Thus, different “first conduits” may be supplied so that each conduit is within a certain range of the end of a container. As addressed herein, the range between the end of a conduit and container helps prevent (fully or partially) the medium (e.g., powder) from being sucked or conveyed into the reservoir. However, as addressed elsewhere herein, other embodiments may use a single “first conduit” to couple to differently sized containers. For example, the conduit may have markings or perforations to guide severance of the conduit at predetermined positions such that the same conduit is adjustable to work with variously sized containers.

However, in other embodiments the distance may be different. The above is just one example and claims not specifically reciting a specific dimension should not be limited to any specific dimension or range.

Example 11d. The system of example 6d comprising the container, wherein: the container is coupled to the fluid reservoir; the container includes first (210) and second (211) container ends that oppose each other; the first conduit does not traverse the first container end; and the first conduit does not traverse the second container end.

Thus, in various embodiments containers of product may be packaged separately from system components such as the fluid reservoir, first and second conduits, and housing. Further, when the container is included in the same packaging as system components such as the fluid reservoir, first and second conduits, the container may or may not be already coupled to system components such as the fluid reservoir, first and second conduits.

Example 12d. The system of example 6d comprising the container, wherein the container is coupled to the fluid reservoir. The container includes first (210) and second (211) container ends that oppose each other. The first conduit traverses the first container end. The first conduit does not traverse the second container end.

Example 13d. The system of example 5d, wherein the flowable medium includes a powder and the fluid includes air.

As used herein, “powder” includes any substance reduced to a state of fine, loose particles.

Such air may include, for example, ambient or typical atmospheric air taken from the surroundings that are immediately adjacent the system.

Example 14d. The system of example 13d, wherein the powder includes a therapeutic agent.

The therapeutic agent may include, for example, collagen.

Example 15d. The system of example 1d, wherein the second conduit traverses the fluid reservoir.

Example 16d. The system of example 15d, wherein the fluid reservoir is between the housing and the second end of the second conduit.

Example 17d. The system of example 16d, wherein the housing includes a shelf (220). The shelf may have a curved surface configured to directly contact the flowable medium. The second channel is between the shelf and the second conduit. The second conduit includes a long axis that traverses the first and second ends of the second conduit and does not contact sidewalls of the second conduit. The long axis of the second conduit intersects the second channel and the shelf.

For instance, in FIG. 7D the curved nature of the shelf is shown. The shelf helps to guide medium (e.g., powder) from the container towards the second channel. Note how the second channel restricts fluid flow from the container into second conduit. This restriction increases the air pressure at the first end of the conduit to better expel medium out of the second conduit. Further, the narrowed second channel also regulates the amount of medium that at any one time is to be expelled from the system.

Embodiments addressed herein provide a more consistent application experience for the user. For example, a user fully depressing the reservoir of Examples 1D-17D will be more likely to experience similar outputs of medium (e.g., powder) without using an overly expensive or complicated application system.

In an embodiment, the first channel includes a first volume and the second channel includes a second volume that is less than the first volume.

Example 18d. A system (200) comprising a container (203) including a flowable medium. A fluid reservoir (201) is configured to: (a) inspire fluid when the fluid reservoir expands and expire fluid when the fluid reservoir contracts, and (b) couple to the container (203). A housing (212) includes first (213) and second (214) channels. The system includes a first conduit (204). A first end (205) of the first conduit couples a second end (206) of the first conduit to the fluid reservoir. The second end of the first conduit is configured to be included in the container when the container is coupled to the fluid reservoir. The system includes a second conduit (207). A first end (208) of the second conduit couples a second end (209) of the second conduit to the housing. The first channel couples the first end of the first conduit to the fluid reservoir. The second channel couples the first end of the second conduit to the container when the container is coupled to the fluid reservoir. The flowable medium is not included in the fluid reservoir.

Another version of Example 18d. A system (200) comprising a container (203) including a flowable medium. A fluid reservoir (201) is configured to: (a) inspire fluid when the fluid reservoir expands and expire fluid when the fluid reservoir contracts, and (b) couple to the container (203). A housing (212) includes first (213) and second (214) channels. The system includes a first conduit (204). A first end (205) of the first conduit couples a second end (206) of the first conduit to the fluid reservoir. The second end of the first conduit is configured to be included in the container when the container is coupled to the fluid reservoir. The system includes a second conduit (207). A first end (208) of the second conduit couples a second end (209) of the second conduit to the housing. The first channel couples the first end of the first conduit to the fluid reservoir. The second channel couples the first end of the second conduit to the container when the container is coupled to the fluid reservoir.

Example 19d. The system of example 18d, wherein: the container is coupled to the fluid reservoir. The container includes first (210) and second (211) container ends that oppose each other. The first conduit traverses the first container end. The first conduit does not traverse the second container end.

Example 20d. The system of example 19d comprising a fluid path, wherein: the fluid path progresses in serial fashion from the fluid reservoir to the first channel, from the first channel to the first conduit, from the first conduit to a second end of the container, from the second end of the container to the second channel, and from the second channel to the second conduit; the system is configured to convey the flowable medium from the container and out the second end of the second conduit when the container is coupled to the fluid reservoir and in response to contraction of the fluid reservoir.

Example 21d. The system of example 20d comprising an alternative first conduit (204′). A first end of the alternative first conduit is configured to couple a second end of the alternative first conduit to the fluid reservoir. The second end of the alternative first conduit is configured to be included in an alternative container when the alternative container is coupled to the fluid reservoir. The first conduit and the alternative first conduit have different lengths from each other. The container and the alternative container have different volumes from each other.

Example 1e. A system comprising: a fluid reservoir configured to: (a) inspire fluid when the fluid reservoir expands and expire fluid when the fluid reservoir contracts, and (b) couple to a container that includes a flowable medium; a housing including first and second channels; a first conduit, wherein: (a) a first end of the first conduit couples a second end of the first conduit to the fluid reservoir, (b) the second end of the first conduit is configured to be included in the container when the container is coupled to the fluid reservoir; a second conduit, wherein a first end of the second conduit couples a second end of the second conduit to the housing; wherein: (a) the first channel couples the first end of the first conduit to the fluid reservoir; and (b) the second channel couples the first end of the second conduit to the container when the container is coupled to the fluid reservoir.

Example 2e. The system of example 1e comprising a fluid path, wherein the fluid path progresses in serial fashion from the fluid reservoir to the first channel, from the first channel to the first conduit, from the first conduit to a second end of the container when the container is coupled to the fluid reservoir, from the second end of the container when the container is coupled to the fluid reservoir to the second channel, and from the second channel to the second conduit.

Example 3e. The system according to any of examples 1e-2e, wherein the system is configured to convey the flowable medium from the container and out the second end of the second conduit when the container is coupled to the fluid reservoir and in response to contraction of the fluid reservoir.

Example 4e. The system according to any of examples 1e-3e, wherein the housing includes a funnel and the first conduit traverses the funnel.

Example 5e. The system according to any of examples 1e-3e, wherein the container includes a funnel and the first conduit is configured to traverse the funnel when the container is coupled to the fluid reservoir.

Example 6e. The system according to any of examples 4e-5e, wherein: the funnel includes first and second ends that oppose each other; the first end of the funnel has a smaller diameter than the second end of the funnel; the first conduit traverses both of the first and second ends of the funnel when the container is coupled to the fluid reservoir.

Example 7e. The system according to any of examples 1e-6e, wherein the system does not include a valve.

Example 8e. The system according to any of examples 2e-7e, wherein the second end of the first conduit is no more than 5 mm from the second end of the container when the container is coupled to the fluid reservoir.

Example 9e. The system according to any of examples 1e-8e wherein: the first conduit includes a long central axis that traverses the first and second ends of the first conduit; the second conduit includes a long central axis that traverses the first and second ends of the second conduit; and the long central axis of the first conduit is parallel to the long central axis of the second conduit.

Example 10e. The system according to any of examples 1e-9e comprising an alternative first conduit, wherein: (a) a first end of the alternative first conduit is configured to couple a second end of the alternative first conduit to the fluid reservoir, (b) the second end of the alternative first conduit is configured to be included in an alternative container when the alternative container is coupled to the fluid reservoir; the first conduit and the alternative first conduit have different lengths from each other; the container and the alternative container have different volumes from each other.

Example 11e. The system according to any of examples 1e, 3e-10e comprising the container, wherein: the container is coupled to the fluid reservoir; the container includes first and second container ends that oppose each other; the first conduit does not traverse the first container end; and the first conduit does not traverse the second container end.

Example 12e. The system according to any of examples 1e, 3e-10e comprising the container, wherein: the container is coupled to the fluid reservoir; the container includes first and second container ends that oppose each other; the first conduit traverses the first container end; and the first conduit does not traverse the second container end.

Example 13e. The system according to any of examples 1e-12e, wherein the flowable medium includes a powder and the fluid includes air.

Example 14e. The system of example 13e, wherein the powder includes a therapeutic agent.

Example 15e. The system according to any of examples 1e-14e, wherein the second conduit traverses the fluid reservoir.

Example 16e. The system according to any of examples 1e-15e, wherein the fluid reservoir is between the housing and the second end of the second conduit.

Example 17e. The system according to any of examples 1e-8e, 9e-16e, wherein: the housing includes a shelf, the shelf having a curved surface configured to directly contact the flowable medium; the second channel is between the shelf and the second conduit; the second conduit includes a long axis that traverses the first and second ends of the second conduit and does not contact sidewalls of the second conduit; and the long axis of the second conduit intersects the second channel and the shelf.

Example 18e. A system comprising: a container including a flowable medium; a fluid reservoir configured to: (a) inspire fluid when the fluid reservoir expands and expire fluid when the fluid reservoir contracts, and (b) couple to the container; a housing including first and second channels; a first conduit, wherein: (a) a first end of the first conduit couples a second end of the first conduit to the fluid reservoir, (b) the second end of the first conduit is configured to be included in the container when the container is coupled to the fluid reservoir; a second conduit, wherein a first end of the second conduit couples a second end of the second conduit to the housing; wherein: (a) the first channel couples the first end of the first conduit to the fluid reservoir; and (b) the second channel couples the first end of the second conduit to the container when the container is coupled to the fluid reservoir.

Example 19e. The system of example 18e, wherein: the container is coupled to the fluid reservoir; the container includes first and second container ends that oppose each other; the first conduit traverses the first container end; and the first conduit does not traverse the second container end.

Example 20e. The system of example 19e comprising a fluid path, wherein: the fluid path progresses in serial fashion from the fluid reservoir to the first channel, from the first channel to the first conduit, from the first conduit to the second end of the container, from the second end of the container to the second channel, and from the second channel to the second conduit; the system is configured to convey the flowable medium from the container and out the second end of the second conduit when the container is coupled to the fluid reservoir and in response to contraction of the fluid reservoir.

Example 21e. The system according to any of examples 18e-20e comprising an alternative first conduit, wherein: (a) a first end of the alternative first conduit is configured to couple a second end of the alternative first conduit to the fluid reservoir, (b) the second end of the alternative first conduit is configured to be included in an alternative container when the alternative container is coupled to the fluid reservoir; the first conduit and the alternative first conduit have different lengths from each other; the container and the alternative container have different volumes from each other.

The foregoing description of the embodiments of the invention has been presented for the purposes of illustration and description. It is not intended to be exhaustive or to limit the invention to the precise forms disclosed. This description and the examples following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. A system comprising:
a fluid reservoir configured to: (a) inspire fluid when the fluid reservoir expands and expire fluid when the fluid reservoir contracts, and (b) couple to a container that includes a flowable medium;
a housing including first and second channels;
a first conduit, wherein: (a) a first end of the first conduit couples a second end of the first conduit to the fluid reservoir, (b) the second end of the first conduit is configured to be included in the container when the container is coupled to the fluid reservoir;
a second conduit, wherein a first end of the second conduit couples a second end of the second conduit to the housing;
wherein: (a) the first channel couples the first end of the first conduit to the fluid reservoir; and (b) the second channel couples the first end of the second conduit to the container when the container is coupled to the fluid reservoir.

2. The system of claim 1 comprising a fluid path, wherein the fluid path progresses in serial fashion from the fluid reservoir to the first channel, from the first channel to the first conduit, from the first conduit to a second end of the container when the container is coupled to the fluid reservoir, from the second end of the container when the container is coupled to the fluid reservoir to the second channel, and from the second channel to the second conduit.

3. The system of claim 2, wherein the system is configured to convey the flowable medium from the container and out the second end of the second conduit when the container is coupled to the fluid reservoir and in response to contraction of the fluid reservoir.

4. The system of claim 3, wherein the housing includes a funnel and the first conduit traverses the funnel.

5. The system of claim 3, wherein the container includes a funnel and the first conduit is configured to traverse the funnel when the container is coupled to the fluid reservoir.

6. The system of claim 5, wherein:
the funnel includes first and second ends that oppose each other;
the first end of the funnel has a smaller diameter than the second end of the funnel;
the first conduit traverses both of the first and second ends of the funnel when the container is coupled to the fluid reservoir.

7. The system of claim 6, wherein the system does not include a valve.

8. The system of claim 2, wherein the second end of the first conduit is no more than 5 mm from the second end of the container when the container is coupled to the fluid reservoir.

9. The system of claim 1 wherein:
the first conduit includes a long central axis that traverses the first and second ends of the first conduit;
the second conduit includes a long central axis that traverses the first and second ends of the second conduit; and
the long central axis of the first conduit is parallel to the long central axis of the second conduit.

10. The system of claim 1 comprising an alternative first conduit, wherein: (a) a first end of the alternative first conduit is configured to couple a second end of the alternative first conduit to the fluid reservoir, (b) the second end of the alternative first conduit is configured to be included in an alternative container when the alternative container is coupled to the fluid reservoir;
the first conduit and the alternative first conduit have different lengths from each other;
the container and the alternative container have different volumes from each other.

11. The system of claim 1 comprising the container, wherein:
the container is coupled to the fluid reservoir;
the container includes first and second container ends that oppose each other;
the first conduit does not traverse the first container end; and
the first conduit does not traverse the second container end.

12. The system of claim 1 comprising the container, wherein:
the container is coupled to the fluid reservoir;
the container includes first and second container ends that oppose each other;
the first conduit traverses the first container end; and
the first conduit does not traverse the second container end.

13. The system of claim 1, wherein the flowable medium includes a powder and the fluid includes air.

14. The system of claim 13, wherein the powder includes a therapeutic agent.

15. The system of claim 1, wherein the second conduit traverses the fluid reservoir.

16. The system of claim 15, wherein the fluid reservoir is between the housing and the second end of the second conduit.

17. The system of claim 16, wherein:
the housing includes a shelf, the shelf having a curved surface configured to directly contact the flowable medium;
the second channel is between the shelf and the second conduit;
the second conduit includes a long axis that traverses the first and second ends of the second conduit and does not contact sidewalls of the second conduit; and
the long axis of the second conduit intersects the second channel and the shelf.

18. A system comprising:
a container including a flowable medium;
a fluid reservoir configured to: (a) inspire fluid when the fluid reservoir expands and expire fluid when the fluid reservoir contracts, and (b) couple to the container;
a housing including first and second channels;
a first conduit, wherein: (a) a first end of the first conduit couples a second end of the first conduit to the fluid reservoir, (b) the second end of the first conduit is configured to be included in the container when the container is coupled to the fluid reservoir;
a second conduit, wherein a first end of the second conduit couples a second end of the second conduit to the housing;
wherein: (a) the first channel couples the first end of the first conduit to the fluid reservoir; and (b) the second channel couples the first end of the second conduit to the container when the container is coupled to the fluid reservoir.

19. The system of claim 18, wherein:
the container is coupled to the fluid reservoir;
the container includes first and second container ends that oppose each other;
the first conduit traverses the first container end; and
the first conduit does not traverse the second container end.

20. The system of claim 19 comprising a fluid path, wherein:
the fluid path progresses in serial fashion from the fluid reservoir to the first channel, from the first channel to the first conduit, from the first conduit to the second end of the container, from the second end of the container to the second channel, and from the second channel to the second conduit;
the system is configured to convey the flowable medium from the container and out the second end of the second conduit when the container is coupled to the fluid reservoir and in response to contraction of the fluid reservoir.

21. The system of claim 20 comprising an alternative first conduit, wherein: (a) a first end of the alternative first conduit is configured to couple a second end of the alternative first conduit to the fluid reservoir, (b) the second end of the alternative first conduit is configured to be included in an alternative container when the alternative container is coupled to the fluid reservoir;
the first conduit and the alternative first conduit have different lengths from each other;
the container and the alternative container have different volumes from each other.
